(19) United States
(12) Patent Application Publication
(10) Pub. No.: US 2005/0169973 A1
(43) Pub. Date: Aug. 4, 2005

(54) Aroma mask pack

(76) Inventor: Young-Ho Kim, Seoul (KR)

Correspondence Address: ST. ONGE STEWARD JOHNSTON & REENS, LLC, 986 BEDFORD STREET, STAMFORD, CT, 06905-5619, US

(21) Appl. No.: 10/783,504

(22) Filed: Feb. 20, 2004

(30) Foreign Application Priority Data
Jan. 29, 2004 (KR) 2004/5570

(51) Int. Cl. A61L 9/00; A61K 35/78

(52) U.S. Cl. 424/443; 424/745; 424/747; 424/764; 424/769

(57) ABSTRACT

The present invention relates to an aroma mask pack that enables cooling massage or hot massage around eyes concurrently with aromatherapy. An aroma mask pack according to the present invention includes: a body formed to cover nose and surroundings of eyes of a face, the body including two porous synthetic resin sheets having openings at locations corresponding to the eyes and peripheries being heat-fused to form an accommodation space therein; a thermal storage medium accommodated in the accommodation space of the body; essential oil mixed with the thermal storage medium and accommodated in the accommodation space of the body; and a wearing means for fixing the body on a face.

### BACKGROUND ART

1. Field of the Invention

The present invention relates to an aroma mask pack, and more particularly, to an aroma mask pack that enables cooling massage or hot massage around eyes concurrently with aromatherapy.

2. Description of the Related Art

Recently, it is known that pure plant oil generating a perfume extracted from a specific plant, that is, essential oil has excellent effects on human physical, mental and psychological therapy. The essential oil is called aromatherapy and is gaining the popularity as alternative medicine in advanced countries.

There are lavender, rosemary, sandalwood, peppermint, chamomile, etc. as the essential oil. The lavender has relieves headache and insomnia. The rosemary enhances mental exercise and relieves myalgia. The sandalwood relieves hypochondria and a feeling of uneasiness. The peppermint relieves digestive troubles. The chamomile relieves alleviation of pain and perspiration, enhances digestion and relieves fatigue. There are inhale, bath, massage, dosage, etc. as the usage of the essential oil.

The inhale is the method of inhaling the perfume emitted from the essential oil by using a nose directly. There are dry inhale and vapor inhale. The dry inhale is the method of dropping a few drops of the essential oil on a handkerchief or gauze and inhaling the smell through a nose. The vapor inhale is the method of dropping a predetermined amount of the essential oil into a hot water and inhaling the perfume escaping with vapor from the hot water with covering a towel on a head.

Since the aromatherapy using inhale can obtain a rapid effect, it is widely used to relieve headache and insomnia and relieve mental and physical fatigue caused by stress. However, since essential oil is dropped on the handkerchief or water, it is consumed too much compared with the amount of actually inhaled perfume and is inconvenient to use.

### SUMMARY OF THE INVENTION

An object of the present invention is to provide an aroma mask pack that can be easily worn on the face, especially, around the eyes to enable aromatherapy and that can maximize a therapy effect by using hot massage and cold massage together if necessary.

According to one aspect of the present invention, an aroma mask pack, comprises: a body formed to cover nose and the surrounding of the eyes of a face, the body including two porous synthetic resin sheets having openings at locations corresponding to the eyes and peripheries being heat-fused to form an accommodation space therein; a thermal storage medium accommodated in the accommodation space of the body; essential oil mixed with the thermal storage medium and accommodated in the accommodation space of the body; and a wearing means for fixing the body on a face.

According to a result of manufacturing and testing an aroma mask pack, the thermal storage medium cannot penetrate fine holes of a porous synthetic resin body and is accommodated in an accommodation space but only fine smell molecules are continuously emitted through fine holes. Accordingly, an handkerchief, hot water, essential oil, etc. are not required additionally and a user easily wears the aroma mask pack on his or her face so that aromatherapy can be performed and a user can work, watch TV or sleep with wearing the aroma mask pack.

If necessary, the body is heated with boiling water or frozen in a refrigerator and the user wears the aroma mask pack, so that the cold massage and the hot massage can be used simultaneously using heat stored in the thermal storage medium and healing effect can be maximized.

A wearing means can be a separate elastic band fixed to the body but is desirably formed integrated with the body.

It is desirable that the thermal storage medium is a mixture of propylene glycol, carboxymetyl cellulose, and sodium benzonate.

It is desirable that the essential oil is any one selected from the group consisting of lavender, rosemary, sandalwood, peppermint, chamomile, and mixture thereof.

### BRIEF DESCRIPTION OF THE DRAWINGS

The above objects and other advantages of the present invention will become more apparent by describing in detail preferred embodiments thereof with reference to the attached drawings in which:

FIG. 1 is a front view of an aroma mask pack according to an embodiment of the present invention;

FIG. 2 is a cross-sectional view taken along the line A-A in FIG. 1;

FIG. 3 is a perspective view illustrating that a man is wearing the aroma mask pack; and

FIG. 4 is a front view of an aroma mask pack according to an embodiment of the present invention.

### DETAILED DESCRIPTION OF THE PREFERRED EMBODIMENT

Now, preferred embodiments of the present invention will be described in detail with reference to the accompanying drawings.

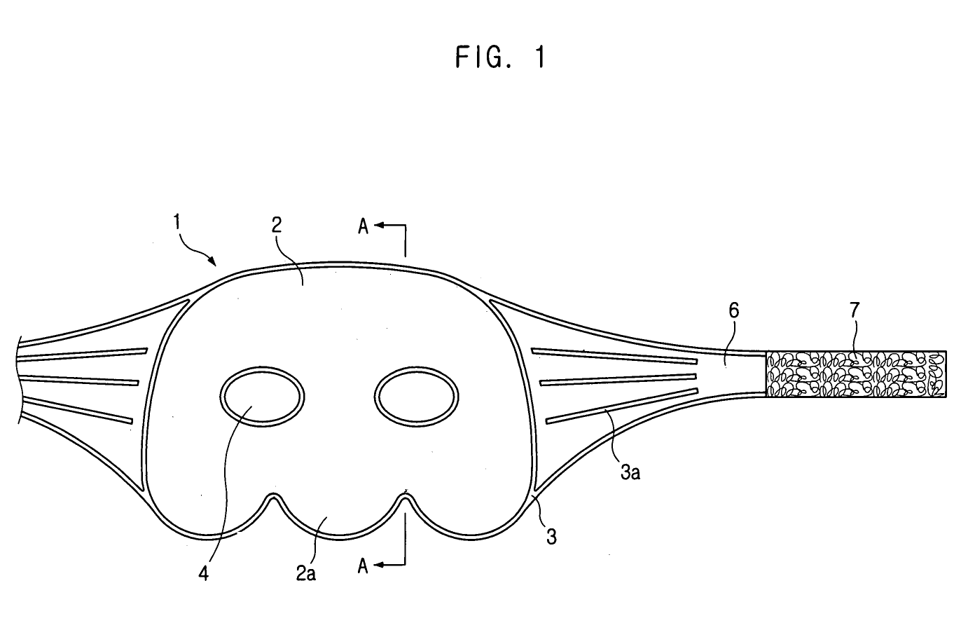

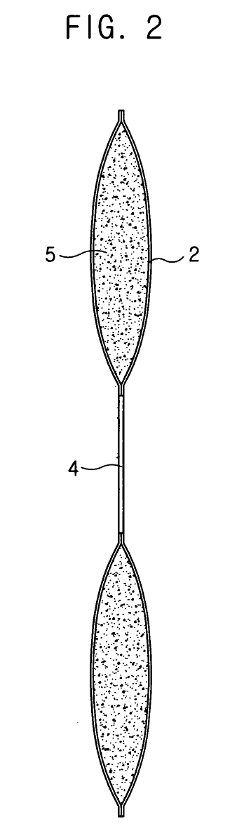

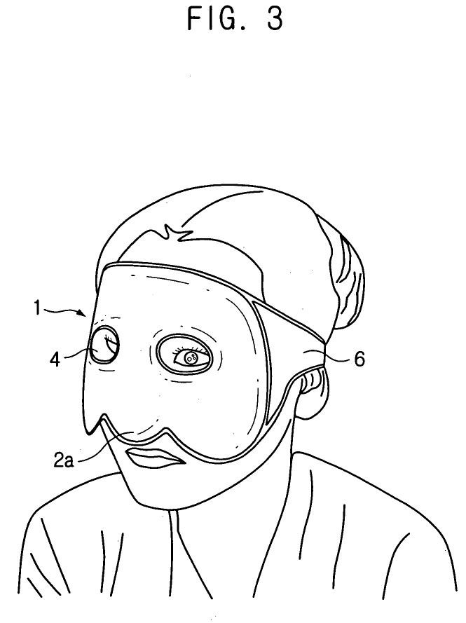

FIG. 1 is a front view of an aroma mask pack according to an embodiment of the present invention. FIG. 2 is a cross-sectional view taken along the line A-A in FIG. 1. FIG. 3 is a perspective view illustrating that a man is wearing the aroma mask pack.

The body 2 of the aroma mask pack 1 is made of two porous soft PVC sheets with lots of fine holes that do not allow solid and liquid to penetrate the body 2 but allow only fine gas molecules to penetrate the body 2. The two porous soft PVC sheets are cut to have openings 4 at the locations corresponding to eyes to cover the surroundings of the eyes and a nose cover 2a extending to the below of the nose to cover the nose. The peripheries of the two sheets are heat-fused to each other to make accommodation space therein.

The accommodation space of the body 2 is filled with mixture of the thermal storage medium and the essential oil. When heat-fused portion 3 is made, the peripheries of the two sheets are heat-fused to have such long slit that the mixture can be injected into the accommodation space. After the mixture is filled in the accommodation space, the slit is heat-fused to seal the accommodation space.

Desirably, the thermal storage medium is a material that can accumulate and store a large amount of heat, especially, the thermal storage medium of gel state-made of mixture of propylene glycol, carboxymetyl cellulose, and sodium benzonate. Therefore, it can be found that the wearing feeling of the aroma mask pack is excellent at both cold state and hot state. Any one selected from the group consisting of the lavender, the rosemary, the sandalwood, the peppermint, the chamomile, and mixture thereof can be used according to application of goods.

Components of the thermal storage medium and essential oil are preferably mixed at a mixing ratio of 60-75% propylene glycol, 7-13% carboxymetyl cellulose, 7-13% sodium benzonate and 7-15% essential oil.

On the other hand, in this embodiment, a band 6 is elongated to both sides of the body 2, and Velcro tapes are fixed to the end of the both sides of the band 6. FIG. 3 illustrates an embodiment in which the body is worn on the nose and the surroundings of the eyes. The band 6 is cut to be integrated with the body 2 when the body 2 is made. The band 6 is heat-fused together with the body 2 when the body 2 is heat-fused. It is better that the peripheries and middle portion 3′ of the band 6 are heat-fused compared with heat-fusing overall portion of the band 6 in the view point of appearance and strength.

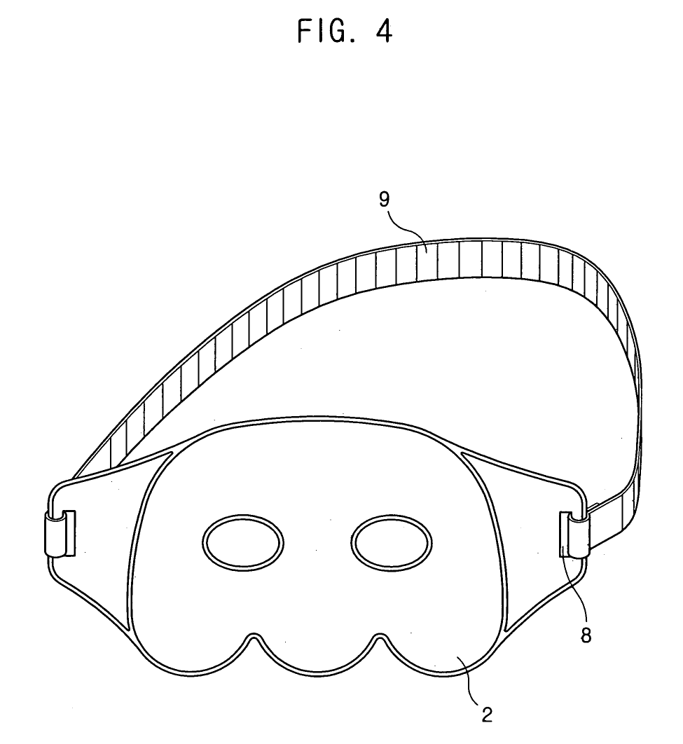

FIG. 4 is a front view of an aroma mask pack according to another embodiment of the present invention. In the embodiment of the present invention, the additional elastic band 9 is fixed to the body. The body 2 is provided with band locking slots on both sides of the body 2 to fix the elastic band to the body 2.

As described above, according to the aroma mask pack of the present invention, the thermal storage medium is not allowed to penetrate the fine holes of the body of porous synthetic resin and is accommodated in the accommodation space. Fine smell molecules are continuously emitted through the fine holes. Since the perfume emitted through the body formed to cover the nose can be absorbed though the nose directly, the handkerchief, the hot water and the essential oil do not have to be prepared additionally and a user can easily wear the aroma mask pack on his or her face, so that aromatherapy can be performed and a user can work, watch TV or sleep with wearing the aroma mask pack.

If necessary, the body is heated with the boiling water or frozen in the refrigerator and the user wears the aroma mask pack, so that cold massage and hot massage can be used simultaneously using heat stored in a thermal storage medium and the healing effect can be maximized.

While the present invention has been described in detail, it should be understood that various changes, substitutions and alterations can be made hereto without departing from the spirit and scope of the invention as defined by the appended claims.

1. An aroma mask pack, comprising:
a body formed to cover nose and surroundings of eyes of a face, the body including two porous synthetic resin sheets having openings at locations corresponding to the eyes and peripheries being heat-fused to form an accommodation space therein;
a thermal storage medium accommodated in the accommodation space of the body;
essential oil mixed with the thermal storage medium and accommodated in the accommodation space of the body; and
a wearing means for fixing the body on a face.

2. The aroma mask pack of claim 1, wherein the thermal storage medium is a mixture of propylene glycol, carboxymetyl cellulose, and sodium benzonate.

3. The aroma mask pack of claim 1, wherein the essential oil is any one selected from the group consisting of lavender, rosemary, sandalwood, peppermint, chamomile, and mixture thereof.
